(19) United States
(12) Patent Application Publication
(10) Pub. No.: US 2026/0301164 A1
(43) Pub. Date: Oct. 1, 2026

(54) MACHINE LEARNING SYSTEM FOR DIAGNOSING BREAST LESIONS, ADAPTABLE TO THE CLINICIAN AND BASED ON ASSERTIVENESS

(71) Applicant: INSTITUTO SUPERIOR TÉCNICO, Lisboa (PT)

(72) Inventors: Francisco Maria GALAMBA FERRARI CALISTO, Lisboa (PT); Carlos Jorge ANDRADE MARIZ SANTIAGO, Lisboa (PT); Jacinto Carlos MARQUES PEIXOTO NASCIMENTO, Lisboa (PT); Duarte Nuno JARDIM NUNES, Lisboa (PT)

(21) Appl. No.: 19/477,964

(22) PCT Filed: Apr. 23, 2024
(86) PCT No.: PCT/PT2024/050013

§ 371 Date: Oct. 23, 2025

(30) Foreign Application Priority Data
Apr. 24, 2023 (PT) 118618

(51) Int. Cl. G06T 7/00

(52) U.S. Cl. CPC G06T 7/0012; G06T 2207/10088; G06T 2207/10132; G06T 2207/20081; G06T 2207/30068

(57) ABSTRACT

The present disclosure relates to the medical imaging field, specifically, associated computer systems for identifying breast lesions. The subject of the present disclosure is a machine learning system for diagnosing breast lesions (10), comprising a first database (11) a second database (12) and at least one server (20) interconnected with the first and second databases, that trains (110) and uses classifier agents (140) to determine a classification and respective segmentation associated with breast lesions through the determination of probable physical parameters with the determination of a probability of occurrence, based on digital images of a breast section (120). The computational determination of probable physical parameters is based adapted on demographic characteristics of the clinician (130), thus implementing a solution with varying degrees of assertiveness (150). This solution improves on known solutions by a determining probability degree of breast lesions existence through a highly adaptable computer system trained for this purpose.

### TECHNICAL FIELD

The present disclosure is in the field of medical imaging, specifically imaging and associated computer systems aimed at identifying breast lesions, thus identifying potential breast cancer lesion masses or potential breast cancer lesion microcalcifications.

### BACKGROUND

Breast cancer is the most common cancer in women worldwide, with almost 1.7 million new cases diagnosed in 2012, representing 12% of new cancers and 25% of all 166 types of cancer in women, according to the article “The efficacy of using computer-aided detection (cad) for detection of breast cancer in mammography screening: a systematic review”, Henriksen, E. et al, Acta Radiologica, 2018.

Medical imaging diagnosis is a routine procedure carried out by radiologists to help diagnose or monitor a medical condition. Medical imaging diagnosis allows doctors to identify pathologies by decoding tissue characteristics by examining features in medical images. It plays a key role in modern medicine, particularly in the prevention and diagnosis of cancer, which is one of the leading causes of mortality worldwide.

The Portuguese patent application with publication number PT 116801 discloses a computational method for the improved identification of breast lesions from digital images obtained using different technologies.

The solution of the present disclosure improves on that solution in that it determines a degree of probability of the existence of breast lesions through a highly adaptable computer system trained for this purpose.

### DISCLOSURE SUMMARY

The subject of the present disclosure is therefore a machine learning system for diagnosing breast lesions, adaptable to the clinician and based on assertiveness, using one or more computing processors.

The system can include:
a first operable database to maintain a diversity of characteristics obtained from digital images of breast sections comprising annotated patterns, at least part of the annotated patterns being associated with breast lesions;
a second operable database to maintain a diversity of demographic characteristics of clinicians;
at least one server interconnected with the first database and the second database, the at least one server being configured to:
   train one or more classifier computer agents on the basis of the annotated patterns in the respective first database;
   computationally obtain one or more digital images of a breast section;
   obtain one or more demographic characteristics of a clinician from a computational interaction provided by the clinician and the diversity of demographic characteristics of clinicians present in the respective second database;
   use one or more classifier agents to computationally determine a classification and respective segmentation associated with one or more breast lesions by determining one or more probable physical parameters, the probable physical parameters comprising potentially existing physical parameters for which a probability of occurrence is determined;
in which the computational determination of probable physical parameters is adapted based on the demographic characteristics of the clinician.

Using one or more classifier computer agents trained on the basis of the annotated patterns in the first database not only improves the determination made by these classifier agents, but also makes the probabilities of occurrence of potentially existing physical parameters more reliable. In addition, it permits a component of assertiveness, in which the computational determination of probable physical parameters and the corresponding availability of this determination are adapted based on the demographic characteristics of the clinician who is interacting with the system. The solution of the present disclosure also enables the detection of breast lesions by adapting the tone of assertiveness.

Demographic characteristics can include a clinician's degree of experience, defined on the basis of a number of years of experience.

Also included in the present disclosure is a system composed of machine learning for diagnosing breast lesions, adaptable to the clinician and based on assertiveness and using one or more computer processors, and consisting of the machine learning system for diagnosing breast lesions of the present disclosure, and at least one piece of imaging equipment.

### DESCRIPTION OF THE FIGURES

FIG. 1—schematic representation of a system (10) according to the present disclosure, comprising a first database (11), a second database (12) and at least one server interconnected with the first database and the second database (20). The server is configured to train one or more classifier computational agents based on the annotated patterns from the respective first database (110), computationally obtain one or more digital images of a breast section (120), obtain one or more demographic characteristics of a clinician from a computational interaction provided by the clinician and from the plurality of demographic characteristics of clinicians present in the respective second database (130), and using the one or more classifier agents to computationally determine a classification and a respective segmentation associated with one or more breast lesions by determining one or more probable physical parameters, the probable physical parameters comprising potentially existing physical parameters for which a probability of occurrence is determined (140), wherein the computational determination of the probable physical parameters is adapted based on the demographic characteristics of the clinician (150).

FIG. 2—digital image of a breast obtained by mammography imaging (MG). A result obtained from the machine learning system for diagnosing breast lesions is shown, which indicates that three potential lesions have been identified, with the type of distribution of each one and some probable physical parameters and additional parameters. The accuracy and a classification according to the Breast Imaging Reporting and Data System (BIRADS) are also presented.

FIG. 3—examples of different presentations of information resulting from the determinations of this disclosure system, with different levels of assertiveness.

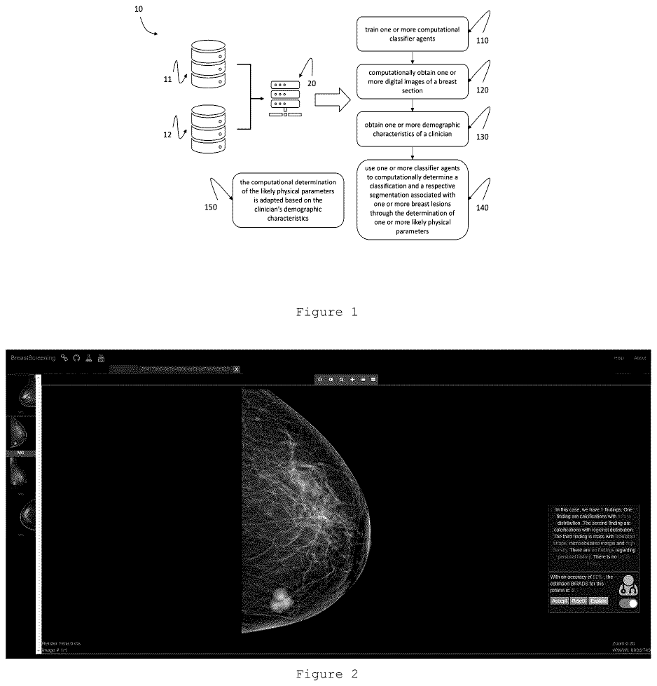

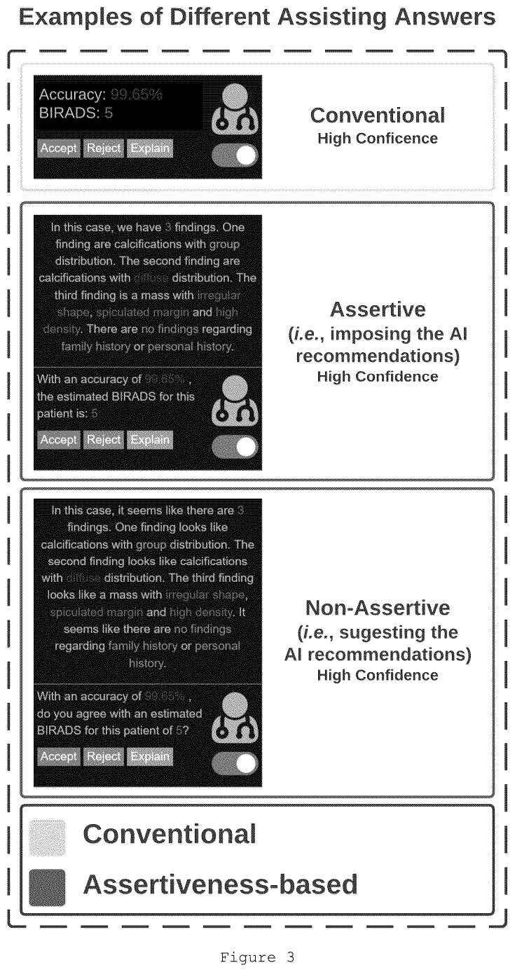

### DETAILED DESCRIPTION

Additional details of the clinician-adaptable, assertiveness-based machine learning system for diagnosing breast lesions of the present disclosure are described below.

The system can be configured for classification according to the Breast Imaging Reporting and Data System (BIRADS).

The system can be configured so that the probable physical parameters include one or more of the following: the number of breast lesions identified, the severity associated with the breast lesions identified, the medical imaging technology of one or more digital images, the visual representation of the breast lesions identified.

The system can also be configured to computationally obtain more than one digital image of the same breast section, these digital images being obtained using different imaging technologies.

The system can also be configured so that the probable physical parameters and their probability are determined for each imaging technology. This allows a more refined determination of any discrepancies between different technologies, when different technologies are used.

The system can also be configured to provide a computational determination of additional physical parameters, in which the computational determination of additional physical of the clinician, the additional physical parameters involving the sensitivity and specificity of one or more of the machine learning models used, and a patient's medical history associated with the digital images and that patient's family members.

The assertiveness component is thus improved such that it also includes additional physical parameters.

The system can be further configured so that the demographic characteristics of the clinician comprise a degree of experience of the clinician, defined on the basis of the number of years of experience of the clinician. Specifically, the system can be further configured so that the demographic characteristics of the clinician comprise a degree of experience of the clinician and include the definition of ranges of clinician years of experience, where these ranges correspond to the experience categories of intern, junior, intermediate, and senior.

As mentioned, the computational determination of the probable physical parameters is adapted based on the demographic characteristics of the clinician.

The determination and potential subsequent presentation of the determination to the clinician could involve determining the parameters density composition, shape and margin of a breast lesion that may consist of a mass, as well as the parameters morphology/type and distribution of a breast lesion that might consist of calcification. With this information, the system can adapt the determination and subsequent presentation of a communication based on an assertive tone for the detection, classification and segmentation of breast lesions.

These one or more digital images can be obtained from mammography, ultrasound and/or magnetic resonance imaging. The system is therefore configured accordingly, so that it can operate with digital images obtained from mammography, ultrasound and/or magnetic resonance imaging.

The system can be configured so that this segmentation is carried out using a geometric shape.

The geometric shape can consist of one or more polygons, eccentric sections or combinations thereof.

Additional details of the composite breast lesion identification system of the present disclosure are described below.

The system may comprise at least two pieces of equipment with different imaging technologies.

Said equipment can be mammography imaging, ultrasound and/or magnetic resonance imaging.

### EXAMPLES

Additional details of the systems in this disclosure are described below.

The machine learning system for diagnosing breast lesions in the present disclosure uses intelligent agents with interactivity capabilities and assertiveness-based Assertiveness is communication for personalised medicine. adapted based on the clinical experience of the clinician/doctor, where the agent adapts a more or less assertive tone, as well as behaving in a more or less reactive/proactive way, depending on the doctor's clinical experience.

Depending on the professional experience of clinicians/doctors (i.e. interns, junior, intermediate and senior), various machine learning/artificial intelligence (AI) results will inform and explain the following to doctors: (1) number of findings detected; (2) patient severity for each breast and by medical imaging modality (e.g. mammography-MG; ultrasound-US; and magnetic resonance imaging-MRI); (3) visual representation of benign or malignant estimates; (4) visualisation of the models' sensitivity and specificity results; and (5) patient's personal and family histories.

As mentioned above, the subsequent presentation of the determination to the clinician may involve determining the parameters density composition, shape and margin of a breast lesion that may consist of a mass, as well as the parameters morphology/type and distribution of a breast lesion that could consist of calcification. With this information, the system can adapt the determination and subsequent presentation of a communication based on an assertive tone for the detection, classification and segmentation of breast lesions.

As an example, a less assertive recommendation will indicate that, according to the probable physical parameters determined, a diagnostic recommendation should be followed. An example would be the indication that “with these determinations, it seems that you should follow my recommendations”. A more assertive recommendation will indicate that, according to the probable physical parameters determined, it is certain that a diagnostic recommendation should be followed. An example would be the statement “with these determinations, I am certain that you should follow my recommendations”.

Through an interface, the patient's classified data from the AI results is communicated by the intelligent agent. The intelligent agent communicates the results in a more assertive (declarative) or less assertive (suggestive) tone, depending on the professional experience of each clinician. Diagnostic data representing the severity of each patient's breast is retrieved. The intelligent agent guides the patient's particular diagnosis for each clinician, adapting the communication narrative via the interface.

The assertiveness-based agent uses recommendations to classify and segment: (1) the number of lesions detected; (2) the severity for each breast of the patient and by medical imaging modality; (3) a visual scale representing benign or malignant estimates; (4) visualisation provide of the sensitivity and specificity results of the models; and (5) with clinical arguments from the patient, such as pathological covariates. To compare the assertiveness-based agent with a conventional agent, participants are given the information that the recommendations are generated by AI models, so that they can also provide some feedback on the model's performance. As mentioned, the subsequent presentation of the determination to the clinician may involve determining the parameters mentioned above.

Applied machine learning/AI models may be known from the state of the art, being distinct at least insofar as they have been trained according to the solution of the present disclosure.

The agent has the ability to recommend classification and segmentation based on the AI model for 2D imaging modalities such as mammography and ultrasound, as well as AI models for 3D imaging modalities such as MRI. The intelligent AI agent provides the classification of the patient's breast severity via the BIRADS scale, the accuracy of the model for this classification and the segmentation of the lesion to explain the regions derived from this classification.

With this system, doctors can access a patient's information by selecting the ID from a list of patients. Once they have accessed the patient's information, doctors can select each respective image of the breast by dragging and dropping each image into the viewing windows. From here, doctors can manipulate the image using the toolbox. The system allows the doctor to accept or reject the AI agent's final recommendation, while clinicians can also request an explanation to support the final decision they take. A typical output from an AI model includes not only the predicted BIRADS classification, but also a probability distribution regarding all possible classification choices.

In this assertiveness-based condition, the agent transmits the clinical arguments along with the communication of BIRADS and accuracy, but this time by descriptive information, such as the parameters mentioned above. Therefore, the assertiveness-based agent is able to communicate confidence quantitatively based on a sentence describing the clinical arguments. Specifically, the image viewing window has been augmented with an additional bounding box or circular ellipse highlighting the lesion characteristics that will explain the final BIRADS classification.

Detailed patient augmentation for medical images in breast cancer diagnosis has been extended with a recommendation and consequent explanation based on assertiveness.

The system set out in the present disclosure has improved how to reliably determine and list the clinical arguments that are later interpretable by human clinicians through the classification and segmentation recommendations given by AI.

This system adapts its communication depending on the demographic characteristics of the doctor, personalising and customising the AI's response to clinicians, as described above.

These clinical arguments correspond to the classification outputs of an AI model, which have been trained on multimodal clinical case data.

As an example, the machine learning system for diagnosing breast lesions that is adaptable to the clinician and based on assertiveness of the present disclosure is capable of determining and presenting a more suggestive or assertive communication tone, depending on specific demographic characteristics of clinicians.

Based on the generic phrase, which contains probable physical parameters:
In this case, we have N findings. A finding is an oval-shaped mass with a circumscribed margin . . . .

One finding will be a breast lesion.

In a Non-Assertive case, the same information will consist of:
In this case, it seems there are N findings. One finding looks like an oval-shaped mass with a circumscribed margin . . . with these findings, it looks like you should follow my recommendations for BIRADS=4 . . . .

In an Assertive case, the same information will consist of:
In this case, we have N findings. One finding is an oval-shaped mass with a circumscribed margin . . . with these findings, I'm sure you should follow my recommendations for BIRADS=4 . . . .

As mentioned, the agents can thus determine probable physical parameters of the lesions, which could take the form of lexical variables of the lesion identified.

If the imaging technology is mammography and the breast lesion is a mass, the lexical variables are as follows.

MASSES

| Shape | Margin | Density |
|---|---|---|
| Round | Circumscribed | High density |
| Oval- Irregular | Obscure | Equal density |
| | Microlobulated | Low density |
| | Indistinct | Containing fat |
| | Spiculated | |

If the imaging technology is mammography and the breast lesion is a calcification, the lexical variables are as follows.

CALCIFICATIONS

Type

| Typically benign | Suspicious morphology | Distribution |
|---|---|---|
| Skin | Amorphous | Diffuse |
| Vascular- Coarse/ | Coarse-heterogeneous | Regional |
| “popcorn-like” | Pleomorphic fine | Grouped |
| Large rod-like | Fine-linear/fine- | Linear |
| Round | linear branching | Segmental |
| Rim- Dystrophic | | |
| Calcium milk | | |
| Suture | | |

If the imaging technology is ultrasound and the breast lesion consists of a mass, the lexical variables are as follows.

MASSES

| | Orienta- | | | Posterior |
|---|---|---|---|---|
| Shape | tion | Margin | Echo pattern | features |
| Oval | Parallel | Circumscribed | Anechoic | No posterior |
| Round | Not | Not | Hyperechoic | features |
| Irregular | parallel | circumscribed: | Complex | Enhancement |
| | | indistinct | cystic and | Shadowing |
| | | angular | solid | Combined |
| | | microlobulated | Hypoechoic | pattern |
| | | spiculated | Isoechoic | |
| | | | Heterogeneous | |

If the imaging technology is magnetic resonance imaging and the breast lesion is a mass, the lexical variables are as follows.

MASSES

| Shape | Margin | Internal enhancement features |
|---|---|---|
| Oval | Circumscribed | Homogeneous |
| Rounded | Uncircumscribed: | Heterogeneous |
| Irregular | Irregular | Rim enhancement |
| | Spiculated | Dark internal septations |

The system, server, databases, agents and/or their elements include components for executing at least some of the feature examples and outcomes of the methods described, whether by means of hardware components (such as memory and/or processor), software or any combination thereof.

The system, server, databases, agents and/or their elements can include general controllers or specific controllers, such as a communications controller, which in turn can consist of or include a microcontroller, capable of receiving external data via inputs and providing triggers for outputs, based on configured rules and/or predefined programming.

An article for use with the system, server, databases, agents and/or elements thereof, such as a pre-recorded storage device or other similar computer-readable medium, including program instructions recorded thereon, or a computer data signal carrying computer-readable program instructions may direct a device to facilitate the implementation of the methods described herein. It is understood that such apparatus and computer data signals are also within the scope of the present disclosure.

A “computer-readable medium” means any medium that can store instructions for use or execution by a computer or other computing device, including read-only memory (ROM), erasable programmable read-only memory (EPROM) or flash memory, random access memory (RAM), a portable floppy disc, a hard disk drive (HDD), a solid state storage device (e.g. NAND flash or synchronous dynamic RAM (SDRAM)) and/or an optical disc, such as a Compact Disc (CD), Digital Versatile Disc (DVD) or Blu-Ray™ Disc.

As will be evident to an expert in the field, this disclosure should not be limited to the details described in this document, and various changes are possible which remain within the scope of this disclosure.

Of course, the details presented above can be combined in different ways, but we will avoid repeating all these combinations here.

1. Machine-learning system for diagnosing breast lesions, adaptable to the clinician and based on assertiveness, and using one or more computer processors, the system being characterised by comprising:
a first operable database to maintain a plurality of characteristics obtained from digital images of breast sections comprising annotated patterns, at least part of the annotated patterns being associated with breast lesions;
a second operable database to maintain a plurality of demographic characteristics of clinicians;
at least one server interconnected with the first database and the second database, the at least one server being configured for:
training one or more classifier computer agents on the basis of the annotated patterns in the respective first database;
computationally obtaining one or more digital images of a breast section;
obtain one or more demographic characteristics of a clinician from a computational interaction provided by the clinician and the plurality of demographic characteristics of clinicians recorded in the respective second database;
use one or more classifier agents to computationally determine a classification and respective segmentation associated with one or more breast lesions by determining one or more probable physical parameters, the probable physical parameters comprising potentially existing physical parameters for which a probability of occurrence is determined;
in which the computational determination of probable physical of the clinician.

2. System according to claim 1 characterised by being additionally configured for the classification to be according to the Breast Imaging Reporting and Data System (BIRADS).

3. System according to claim 1 characterised in that it is configured so that the probable physical parameters include one or more of the following: the number of breast lesions identified, the severity associated with the breast lesions identified, the medical imaging technology of one or more digital images, the visual representation of the breast lesions identified.

4. System according to claim 1 characterised by being configured to computationally obtain more than one digital image of the same breast section, said digital images being obtained using different imaging technologies.

5. System according to claim 4 characterised in that it is additionally configured so that probable physical parameters and their probability are determined for each imaging technology.

6. System according to claim 1 characterised in that it is further configured to provide for a computational determination of additional physical parameters,
in which the computational determination of additional physical of the clinician,
the additional physical parameters comprising the sensitivity and specificity of one or more of the machine learning models used and the medical history of a patient associated with the digital images and of that patient's family members.

7. System according to claim 1 characterised in that it is further configured so that the demographic characteristics of the clinician include a degree of experience of the clinician, defined on the basis of a number of years of experience of the clinician.

8. System according to claim 1 characterised in that it is further configured for the demographic characteristics of the clinician to comprise a degree of experience of the clinician including the definition of ranges of years of experience of the clinician, said ranges corresponding to the experience categories of intern, junior, intermediate and senior.

9. System according to claim 1 characterised by one or more digital images being obtained from mammography, ultrasound and/or magnetic resonance imaging.

10. System according to claim 1 characterised by being configured so that said segmentation is carried out using a geometric shape.

11. System according to claim 10 in which the geometric shape consists of one or more polygons, eccentric sections or combinations thereof.

12. Composite machine learning system for diagnosing breast lesions that is adaptable to the clinician and based on assertiveness and using one or more computer processors characterised by including at least one imaging device and machine-learning system for diagnosing breast lesions,
the machine-learning system for diagnosing breast lesions being i) adaptable to the clinician and based on assertiveness, ii) using one or more computer processors, and iii) comprising:
a first operable database to maintain a plurality of characteristics obtained from digital images of breast sections comprising annotated patterns, at least part of the annotated patterns being associated with breast lesions;
a second operable database to maintain a plurality of demographic characteristics of clinicians;
at least one server interconnected with the first database and the second database, the at least one server being configured for:
training one or more classifier computer agents on the basis of the annotated patterns in the respective first database;
computationally obtaining one or more digital images of a breast section;
obtain one or more demographic characteristics of a clinician from a computational interaction provided by the clinician and the plurality of demographic characteristics of clinicians recorded in the respective second database;
use one or more classifier agents to computationally determine a classification and respective segmentation associated with one or more breast lesions by determining one or more probable physical parameters, the probable physical parameters comprising potentially existing physical parameters for which a probability of occurrence is determined;
in which the computational determination of probable physical parameters is adapted based on the demographic characteristics of the clinician.

13. Composite system according to claim 12 characterised by comprising at least two different imaging technology devices.

14. Composite system according to claim 12 characterised by the said device is mammography, ultrasound and/or magnetic resonance imaging device.
